(19) United States
(12) Patent Application Publication
(10) Pub. No.: US 2009/0130160 A1
(43) Pub. Date: May 21, 2009

(54) FIBER FOR WOUND DRESSING

(75) Inventor: Jeffrey S. Dugan, Erwin, TN (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Fiber Innovation Technology, Inc.

(21) Appl. No.: 12/273,866

(22) Filed: Nov. 19, 2008

Related U.S. Application Data
(60) Provisional application No. 60/989,696, filed on Nov. 21, 2007.
(60) Provisional application No. 61/019,917, filed on Jan. 9, 2008.

(51) Int. Cl. A61K 9/00; A61K 33/38; A61K 9/70

(52) U.S. Cl. 424/407; 424/618; 424/409

(57) ABSTRACT

The invention provides a fluid-wicking, antimicrobial fiber formed of a grooved fiber comprising an antimicrobial material. Fabrics and products comprising the fluid-wicking, antimicrobial fiber are also provided. A medical device of the invention, for example a wound dressing, demonstrates antibacterial and optionally antifungal properties, depending on the selection of the antimicrobial material contained therein, with the medical device demonstrating the ability to wick fluids away from the surface of a wound and hold the fluids within an absorbent layer of the device.

### CROSS REFERENCE TO RELATED APPLICATIONS

This application claims priority to U.S. Provisional Patent Application No. 60/989,696, filed on Nov. 21, 2007, and U.S. Provisional Patent Application No. 61/019,917, filed Jan. 9, 2008, both of which are incorporated by reference herein in their entirety.

### FIELD OF INVENTION

The present invention relates to fibers for use in a wound dressing. More particularly, the invention relates to a fiber having grooved channel structures and comprising an antimicrobial material. A wound dressing comprising the inventive fibers wicks away fluid from the surface of a wound and simultaneously provides the antimicrobial material to the wound.

### BACKGROUND OF THE INVENTION

Dry wounds in mammals heal more slowly than wounds that are kept moist by applied dressings. Maintaining moisture at the wound and surrounding epidermis promotes wound closure. Failing to keep a wound dressing moist causes the dry wound dressing to stick to the wound surface disrupting the cellular growth process needed for wound repair to occur. The lack of moisture causes scabbing, which tends to slow the wound healing process.

On the other hand, a wound that produces an excessive amount of moisture results in skin maceration. Skin maceration is the softening of the epidermal tissue surrounding the wound. The condition causes a breakdown of the cornified epithelium, the skin's natural impervious barrier to foreign materials. The condition also makes the wound more susceptible to contamination by pathogenic microbes that would otherwise be inhibited by the epithilia.

It is known to be useful for wound dressings to have the ability to withdraw excessive wound fluid from the wound and into absorbent layers within the dressing. However, while there are a number of dressings designed to retain the wound exudates, such dressings suffer several deficiencies. For example, these dressings are only effective for moist wounds but do not provide any benefit for wounds that do not naturally generate fluid. Furthermore, wounds that generate fluids do so at different rates depending, for example, on the extent of the wound, location of the wound, and the wounded human or other animal. Indeed, even the amount of fluid generated by a single wound will vary over the healing process. Such deficiencies can create a need to remove dressings often either for purposes of adding fluid to a dry wound or to replace a wound dressing that becomes saturated with fluid. Removal of the dressing tends to disrupt the cellular processes associated with wound repair and can result in contamination of the wound by microbes.

Wounds cause damage to the cornified epithelium, the skin's natural microbial barrier. The loss of this barrier creates the risk of microbial contamination at the wound site, which can also disrupt the healing process. Effective treatment requires preventing wound contamination caused by pathogenic microbes. While many antimicrobial materials are available, these materials have conventionally been applied directly to the wound surface as a topical composition with the dressing. A conventional wound dressing that is designed to wick away fluid from the wound will also inevitably carry away any topically applied antimicrobial agent. Ultimately, at some point during the healing process, the topically applied antimicrobial agent itself will lose its effectiveness if it does not entirely disappear altogether.

While silver is known in the art to be an effective antimicrobial material, the controlled release of silver ions from compounds containing the metal has only been achieved through electrical stimulation. It is known by those skilled in the art that the effectiveness of these antimicrobial delivery systems tends to become reduced over the healing cycle as the dressing becomes saturated with fluids that have been wicked away from the wound and the conductive resistance required for silver ion transport becomes increased. Therefore, the most viable conventional treatment for preventing microbial contamination of wounds is by providing a physical barrier that must be manipulated and interrupted through the course of the healing process, activities themselves that increase the risk of microbial contamination and interrupt the healing process.

While negative pressure therapies have been developed to be used in combination with wound dressings for the treatment of soft tissue damage and wound closure, such therapies cannot easily be applied without the assistance of a trained medical professional and are not amenable to delivery to the general public in an off-the-shelf packaged treatment form.

The ability to transport or wick fluids and to hold fluids are two important features of absorbent dressings. The extent of the fluid transport that can occur in a fibrous structure can be controlled by various factors including the geometry and extent of pore structures within the fabrics for promoting transport through capillary action, the nature of the fibrous surface, the geometry of the fibrous surface, physical/chemical treatment of the fibrous surface, and the nature of the fluid to be transported. The physical structure of the fibers themselves can also play an important role in the fluid transport nature of a wound dressing. For example, fibers having a high affinity for wicking can be well-suited for certain moisture transport applications. However, the use of a wicking fiber alone does not resolve the problem of preventing microbial contamination at the site of a wound.

There remains a need in the art for a wound dressing that controls and maintains the proper moisture conditions at the surface of a wound in a human and/or animal and controls the release of antimicrobial materials to the wound surface over the healing cycle.

### BRIEF SUMMARY OF THE INVENTION

The present invention relates to devices and methods of treating a wound in a human and/or animal. While not wishing to be bound by theory, it is believed the devices and methods of the present invention act to decrease, inhibit, or eliminate the replication or growth of a microbial species at the site of or in the vicinity of a wound while, at the same time, maintaining the proper moisture conditions at the wound surface over the course of the healing process of the wound.

In one aspect, the invention provides a fluid-wicking, antimicrobial fiber. In certain embodiments, the fluid-wicking, antimicrobial fiber has a grooved structure. In a preferred embodiment of the invention, the grooved fiber is a capillary-grooved fiber. The fluid-wicking, antimicrobial fiber also has a cross-section showing a plurality of grooves with the plurality of grooves being substantially continuous along an axial direction of the fiber. The fiber has an outer surface and an interior. The fiber is preferably formed of a polymeric material.

In addition to the grooved fiber, the fluid-wicking, antimicrobial fiber of the invention also comprises an antimicrobial material. The antimicrobial material can be combined with the grooved fiber in a variety of ways. For example, in one embodiment, the antimicrobial material is incorporated into the fiber such that at least a portion of the antimicrobial material is in contact with the outer fiber surface. Thus, the antimicrobial material incorporated into the fiber may be present only as a coating on an exposed surface of the fiber, may be at least partially embedded into an exposed surface of the fiber, may be dispersed throughout a discrete section or portion of the fiber or throughout the fiber generally, or may be incorporated by any combination of such means or other means recognizable as useful in light of the present disclosure.

In another embodiment of the invention, the antimicrobial material comprises a metal. In yet other embodiments, the metal comprises at least one of silver and copper.

In one embodiment of the invention, the antimicrobial material used in the invention is a nanoparticulate material. In yet another embodiment, the nanoparticulate material is a nanoparticle silver.

In certain embodiments of the invention, the polymeric material is a melt-spinnable thermoplastic. In specific embodiments, the polymeric material comprises at least one material selected from the group consisting of nylon 6, nylon 6,6, polyethylene terephthalate, polybutylene terephthalate, polypropylene terephthalate, polylactic acid, polypropylene, polyethylene, and combinations thereof.

In another aspect, the invention provides fabrics that are comprised of the fluid-wicking, antimicrobial fiber of the invention. In certain embodiments, the fabric can be a woven fabric, a nonwoven fabric, a knitted fabric, and combinations thereof. In another embodiment of the invention, the fiber that is part of the fabric is in the form of a filament yarn, a tow, or a staple fiber.

In yet another aspect of the invention, a wound dressing is comprised of a fluid-wicking, antimicrobial fiber of the invention. In yet another aspect of the invention, a wound dressing is comprised of at least one of the fabrics of the invention. In yet another aspect of the invention, at least one layer of a wound dressing is comprised of a fluid wicking, antimicrobial fiber of the invention, the inventive fiber in the form of at least one of a filament yarn, a tow, and a staple fiber.

In yet another aspect of the invention there is provided a bandage that is comprised of a wound dressing of the invention. In an embodiment of the invention, a bandage is further comprised of an adhesive. In an embodiment of the invention, the adhesive is used for adhering the bandage to an area of skin adjacent to a wound of a human and/or animal.

In an aspect of the invention, the wound dressing has a fabric for covering an exposed wound surface, the fabric having a grooved fiber with an outer fiber surface and a cross-section that forms continuous longitudinal grooves along an axial direction of the fiber. In a preferred embodiment of the invention, the grooved fiber is a capillary-grooved fiber. The fiber is formed of a polymeric material having an antimicrobial material incorporated therein such that at least a portion of the antimicrobial material is in contact with the outer fiber surface. Without intending to be bound by theory, the grooved fiber wicks away fluid from the wound surface while simultaneously preventing, reducing, and/or eliminating microbe viability at the wound surface. Wicking ability can preferably be varied by controlling the number of grooves (and the dimensions of the grooves) in the fibers.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 is an illustration of a cross-sectional view of a grooved fiber useful according to certain embodiments of the invention;

FIG. 2 is an illustration, partly in cross-section, of a grooved fiber useful according to certain embodiments of the invention showing the substantially continuous axial longitudinal grooves;

FIG. 3 is an illustration of a cross-sectional view of another grooved fiber useful according to certain embodiments of the invention;

FIG. 4 is an illustration of a cross-sectional view of still another grooved fiber useful according to certain embodiments of the invention;

FIG. 5 is an illustration, partly in cross-section, of a capillary-grooved fiber useful according to certain embodiments of the invention having substantially symmetrical channels;

FIG. 6 is an illustration of a cross-sectional view of an embodiment showing an antimicrobial material disposed within the grooves of a grooved fiber according to one embodiment of the invention;

FIG. 7 is an exemplary process according to one embodiment of the invention for disposing an antimicrobial material into the grooves of a grooved fiber;

FIG. 8 is schematic illustration of an exemplary process for producing a multicomponent fiber according to certain embodiments of the invention;

FIG. 9 is an illustration of a general embodiment of an antimicrobial absorbent structure comprising fibers according to certain embodiments of the invention;

FIG. 10 is an illustration of another embodiment of an antimicrobial absorbent structure comprising fibers according to certain embodiments of the invention;

FIG. 11 shows a representative wound dressing device according to one embodiment of the invention;

FIG. 12 shows another embodiment of the wound dressing according to certain embodiments of the invention having two absorbent layers each having at least one fluid-wicking, antimicrobial fiber of the invention; and

FIG. 13 shows an embodiment of a bandage comprising a wound dressing of the invention.

### DETAILED DESCRIPTION OF THE INVENTION

The present invention now will be described more fully hereinafter with reference to the accompanying drawings, in which some, but not all embodiments of the inventions are shown. Preferred embodiments of the invention may be described, but this invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments of the invention are not to be interpreted in any way as limiting the invention. Like numbers refer to like elements throughout.

Many modifications and other embodiments of the inventions set forth herein will come to mind to one skilled in the art to which these inventions pertain having the benefit of the teachings presented in the descriptions herein and the associated drawings. Therefore, it is to be understood that the inventions are not to be limited to the specific embodiments disclosed and that modifications and other embodiments are intended to be included within the scope of the appended claims.

As used in the specification and in the appended claims, the singular forms “a”, “an”, and “the” include plural referents unless the context clearly indicates otherwise. For example, reference to “a fiber” includes a plurality of such fibers.

It will be understood that relative terms, such as “radially” or “circumferentially” or “bottom” or “top” or the like, may be used herein to describe one element's relationship to another element as illustrated in the Figures. It will be understood that relative terms are intended to encompass different orientations of the articles in addition to the orientation as illustrated in the Figures. It will be understood that such terms can be used to describe the relative positions of the element or elements of the invention and are not intended, unless the context clearly indicates otherwise, to be limiting.

Embodiments of the present invention are described herein with reference to various perspectives, including cross-sectional and perspective views that are schematic representations of idealized embodiments of the present invention. As a person having ordinary skill in the art to which this invention belongs would appreciate, variations from or modifications to the shapes as illustrated in the Figures are to be expected in practicing the invention. Such variations and/or modifications can be the result of manufacturing techniques, design considerations, and the like, and such variations are intended to be included herein within the scope of the present invention and as further set forth in the claims that follow. The articles of the present invention and their respective components illustrated in the Figures are not intended to illustrate the precise shape of the component of an article and are not intended to limit the scope of the present invention.

The present invention provides grooved fibers that comprise a microbicidal or an antimicrobial material. Further disclosed herein are fabrics comprising the inventive antimicrobial fibers. Such fabrics are particularly useful in certain medical products including wound dressings. The invention also provides a variety of products of manufacture that can be made using the antimicrobial fibers and fabrics as described herein. The present invention provides improvements over antimicrobial fibers known in the art and fabrics made therefrom by providing the improved ability to inhibit, decrease, and/or eliminate the replication or growth of a microbial species at the site of or in the vicinity of a wound while, at the same time, maintaining the proper moisture conditions at the wound surface over the course of the wound healing process.

The inventive fiber is useful in a variety of products of manufacture incorporating fabrics and fibers as described herein. Applications where the inventive fibers and fabrics made therefrom can be useful include, but are not limited to, any application where the moisture retaining and antimicrobial properties of the inventive fibers and fabrics are desirable. Such applications include, but are not limited to, medical devices; clothing or other fabrics for use in clean room environments or other environments requiring a high degree of microbial control; fabrics and linens used in restaurants and kitchens; and cosmetic applicators and appliances.

A non-limiting use for the inventive fiber and fabrics made therefrom includes any medical device for the treatment of a wound in a human or any animal. Such a wound can be internal or external to the body of a human or an animal and involve, for example, a tissue, organ, epithelium layer, vein, artery, and the like. Such a wound can include, but is not limited to, unbroken or broken skin, bruises, hematomas, inflammation, lesions, rashes, blisters, pustules, abrasions, hives, dermal eruptions, partial thickness wounds, partial thickness burns, incisions, skin graft sites, skin donor sites, lacerations, Stage I-IV dermal ulcers, venous stasis ulcerations, diabetic ulcers, decubitus ulcers, organ lacerations, diabetic ulcers, decubitus ulcers, organ lacerations, organ tears, or external and internal surgical wounds.

The invention is particularly directed to grooved fibers. The grooved fiber can have a complex geometry due to at least one grooved channel present on the outer surface of the fiber, the groove (also referred to as a channel or a capillary channel) substantially following a longitudinal axial direction of the fiber. A grooved fiber, as used herein, thus refers to a fiber having at least one groove formed at the outer fiber surface. In certain embodiments, a grooved fiber according to the invention comprises a plurality of channels formed at the surface of the fiber. Preferably, the capillary channels each have a plurality of channel walls, a base, an opening to the surrounding environment, and a cavity defined by the plurality of channel walls, the base, and the opening. Preferentially, the capillary channels extend substantially continuous and coextensive with an axial direction of the fiber surface. Moreover, it is preferable for the channel opening to be continuous along the axial direction of the fiber. The channel walls that partially define the grooves can be referred to herein as lobes. Thus, a grooved fiber of the invention can be described as a multi-lobular fiber, wherein the multiple lobes define a series of grooves, or channels, existing between the lobes.

The grooved fibers are particularly useful because of their ability to direct fluid flow along the length of the fiber. Particularly, the grooves can impart a capillary (or wicking) action to the fiber. Such wicking action is at least partially defined by the dimensions of the grooves in the fiber. In certain embodiments, the fibers of the invention can be particularly referred to as capillary-grooved fibers. A capillary groove, as used herein, describes a fiber groove having dimensions that promote capillary action. In certain embodiments, a capillary-groove can encompass a groove wherein the cross-section circumferentially encapsulates more than about 180° of surface curvature as defined by the groove walls (or adjacent lobes). In specific embodiments, a capillary groove is a groove wherein the cross-section circumferentially encapsulates more than about 200°, more than about 220°, more than about 240°, more than about 250°, more than about 260°, more than about 270°, more than about 280°, more than about 290°, or more than about 300° of surface curvature as defined by the groove walls.

A non-limiting example of a commercially available fiber that can be used in the invention includes the 4DG™ fiber available from Fiber Innovation Technology of Johnson City, Tenn. The deep grooves of the 4DG fiber provide the fiber with a high surface area. Furthermore, the channels in the surface of a 4DG fiber promote capillary wicking. As a result, these fibers are exceptionally well-suited for moisture transport applications. Depending on the needs of a particular application, these fibers can be used to move liquid away from a source for efficient evaporation or they can be used to absorb and store the liquid in the medium where the fiber is held.

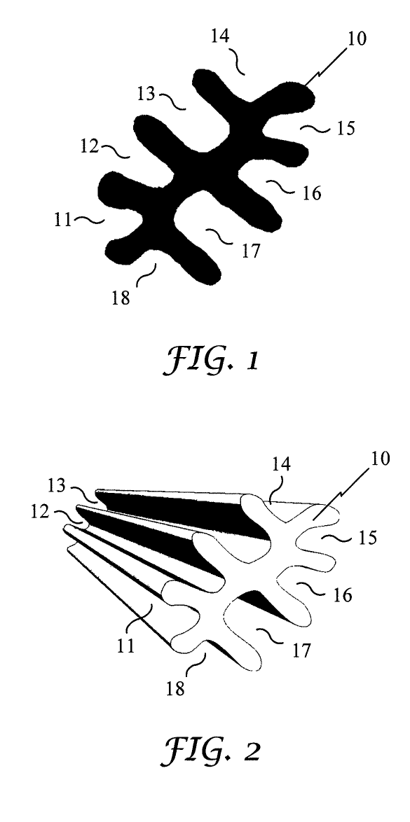

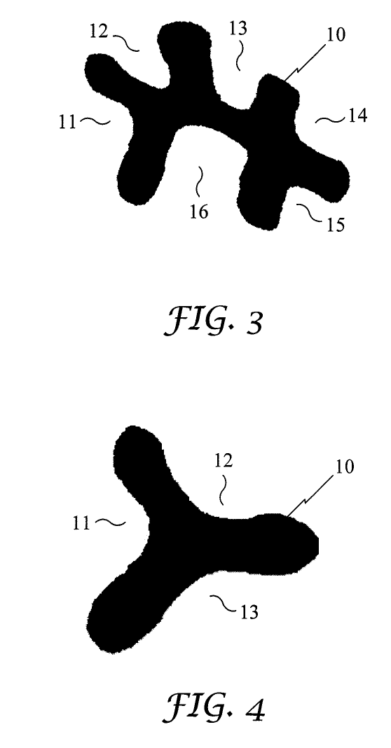

FIG. 1 is a cross-sectional view of one embodiment of a capillary-grooved fiber 10 that is multi-lobular having eight grooves 11-18. FIG. 2 is a three-dimensional view, partly in cross-section, of the same capillary-grooved fiber 10 showing how the grooves 11-18 are substantially continuous along the axial longitudinal length of the fiber. FIG. 3 is a cross-sectional view of a capillary-grooved fiber 10 that is multi-lobular having six grooves 11-16 found in another embodiment of the invention.

As seen in FIG. 1 through FIG. 3, the groove dimensions are such that the grooves would be expected to exhibit a capillary or wicking action. FIG. 4 is a cross-sectional view of a grooved fiber 10 that is multi-lobular having three grooves 11-13 found in yet another embodiment of the invention. In contrast, the grooves of the fiber of FIG. 4 have a much wider dimension than the grooves in the fibers of FIG. 1 through FIG. 3. Accordingly, the fiber of FIG. 4 would be expected to exhibit a reduced wicking or capillary action by comparison.

The grooved fibers used in the inventive fluid-wicking, antimicrobial fibers can have many different conformations. For example, as disclosed in U.S. Pat. No. 6,555,262, incorporated herein by reference, the fiber can be trilobal having radially projecting lobes with the lobes continuing to extend circumferentially in diametrically opposed directions at the outermost surface of the fiber giving a partially enclosed channel. One skilled in the art, having the benefit of the present disclosure, could prepare capillary-grooved fibers having even further conformations, and all such conformations are expressly compassed by the present invention.

The grooved fibers used in certain embodiments of the invention have lobe configurations with asymmetrical channels while the grooved fibers used in other embodiments of the invention have lobe configurations with substantially symmetrical channels. Yet other embodiments include grooved fibers having asymmetrical channels and grooved fibers having symmetrical channels.

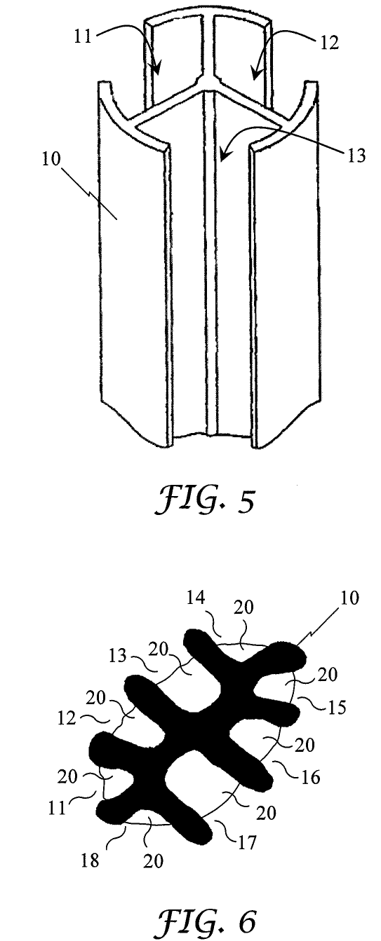

FIG. 5 is a capillary-grooved fiber 10, partly in cross-section, that is multi-lobular having three grooves 11-13 with substantially symmetrical channels found in certain embodiments of the invention. Also useful, in some embodiments of the invention, are the radially projecting lobes with the lobes continuing to extend circumferentially in diametrically opposed directions at the outermost surface of the capillary-grooved fiber giving a partially enclosed channel also shown in FIG. 5. The lobe extensions of this embodiment serve to enhance the capillary action of the grooves.

Defining optimal groove geometry is also important in achieving liquid flux or the rate of liquid transport of a fluid. Liquid flux is related to adhesion tension, which is a variable determined by the surface tension of the fluid and the contact angle the fluid makes with the surface of the groove. The contact angle must be less than 90° in order for the surface to be considered wetted by the fluid. The smaller the contact angle less than 90°, then the higher the adhesion tension will be, a condition that is more favorable to promoting fluid wicking throughout the fiber medium. Hence, the choice of groove width and depth and general alignment of the inventive fibers within the fibrous medium is determinative of whether spontaneous fluid flux is achieved for a given fluid and, if spontaneous flux is achieved, the degree of the flux within the fibrous medium. Furthermore, the rate of liquid transport can relate to groove dimensions. In a preferred embodiment of the invention, a fluid-wicking, antimicrobial fiber is comprised of a capillary-grooved fiber having lobes forming eight grooves similar to the 4DG fiber since this capillary-grooved fiber promotes wicking of fluids generated by human and other animal wounds. A cross-sectional configuration of an example embodiment of the preferred eight groove fiber is illustrated in FIG. 1. In a preferred embodiment, the fluid-wicking, antimicrobial fiber is comprised of a capillary-grooved fiber having lobes forming eight grooves similar to the 4DG fiber.

A more detailed discussion concerning fluid transport properties in wickable fibers can be found in U.S. Pat. No. 5,200,248 to Thompson et al., U.S. Pat. No. 5,972,505 to Phillips et al., U.S. Pat. No. 6,437,214 to Everett et al., and U.S. Pat. No. 6,753,082 to Lobovsky, all incorporated herein by reference.

In addition to the geometry of the fiber surface and the grooves in the capillary-grooved fiber, the extent of the adhesive forces of the fluid with the surface of the fiber is a major determinative factor of whether fluid transport will occur in a fabric comprising a plurality of capillary-grooved fibers. Spontaneous liquid transport will occur when the geometry of the fiber and its corresponding grooves is such that capillary action is favored for the given properties of the fluid that is to be wicked away. Without intending to be bound by theory, capillary action will occur when the adhesive forces between the fluid and the surface of the grooves of the capillary-grooved fiber are stronger than the cohesive forces inside the fluid. The intermolecular force between the fluid and the surface of the grooves is proportional to the surface tension of the fluid or the ability of the fluid to continue to traverse the grooves. A fluid having a relatively high surface tension tends to favor spontaneously liquid transport more so than a fluid having a relatively low surface tension. The type of polymer used in the fiber and the type of fiber surface treatment may also affect the ability of the fluid to effectively be wicked through the grooves of the capillary-grooved fiber. Without intending to be bound by theory, certain surface treatments have the resulting effect of increasing the surface tension of the fluid or, in reality, effectively increasing the adhesive force between the fluid and the surface of the fiber, which tends to promote wicking of the fluid in question.

There are many surface treatment methods known in the art for improving the hydrophilic nature of polymer-based fibers particularly because polymer-based materials tend to have both low polarity and a crystalline structure, each contributing to make the material more hydrophobic than hydrophilic. Surface treatment processes including, but not limited to, chemical and/or solvent processing, ozonization, plasma treatment, UV irradiation processing, high-pressure electro-discharge treatment, corona discharge processing, and flame treatment have been employed to render polymer surfaces more hydrophilic. Furthermore, it is also possible to coat the surface of the polymer with a material that promotes the hydrophilic nature of the fiber. An example of such a surface composition is disclosed in U.S. Pat. No. 7,294,673 to Kanazawa involving a three-step procedure for applying the composition including impregnation, activation, and monomer grafting. Other surface compositions for improving the hydrophilic capability of polymer fibers can be found in U.S. Pat. No. 5,258,129 to Kato et al. (fluid permeable agent for polyolefin fibers), U.S. Pat. No. 5,683,610 to Nohr et al. (use of surfactants to render a hydrophobic polymer fabric wettable), U.S. Pat. No. 5,972,505 to Phillips et al. (hydrophilic surface lubricants including polyoxyethylene, lauryl ether, polyoxyethylene oleyl ether, polyoxylene-polyoxypropylene-sorbitan linoleic phthalic ester, Milease T, and a potassium lauryl phosphate based lubricant), and U.S. Pat. No. 6,359,079 to Palmer (a durable hydrophilic coating composition for polyester, polypropylene, polyethylene, cotton, polyamide, or polyaramid fibers).

Of course, the polymeric material forming the fiber can be made to be more hydrophilic. Non-limiting examples of polymer compositions that are more hydrophilic are disclosed in U.S. Pat. No. 4,814,131 to Atlas (a polymer polyblend including a non-crystalline hydrophilic polymer component), U.S. Pat. No. 5,149,335 to Kellenberger et al. (block copolymers of nylon such as nylon-6 or polyethylene oxide diamine), U.S. Pat. No. 6,045,869 to Gesser et al. (copolymerization of polyhydroxystyrene with polyallylamine, polyaminostyrene, polyacrylamide, or polyacrylic acid), U.S. Pat. No. 6,716,270 to Ding et al. (polyamic acid), U.S. Pat. No. 7,230,043 to Klun et al. (thermoplastic or thermoset polymer with a fluorochemical additive), and U.S. Pat. No. 7,235,592 to Muratoglu et al. (polyvinyl alcohol).

In an embodiment of the invention, a fluid-wicking antimicrobial fiber of the invention is comprised of a polymeric material with any needed surface treatment and/or composition as disclosed herein, to promote the desired rate of fluid flux through the fibrous medium. Preferably, a fluid-wicking antimicrobial fiber of the invention improves the wicking rate of conventional capillary-grooved fibers by at least about 20%, by a least about 40%, by at least about, 60%, by at least about 80%, by at least about 100%, and more.

The grooved fiber of the invention can be formed of any polymeric material useful in forming a fiber, such as through conventional extrusion techniques. In an embodiment of the invention, the polymeric material is a melt-spinnable thermoplastic polymer. In a preferred embodiment of the invention, the grooved fiber is formed of a polymeric material comprising at least one material selected from the group consisting of nylon 6, nylon 6,6, polyethylene terephthalate, polybutylene terephthalate, polypropylene terephthalate, polylactic acid, polypropylene, polyethylene, and combinations thereof.

In specific embodiments, polymers used in the inventive fibers comprise functional groups making them particularly compatible with the antimicrobial materials used with the fibers. Preferably, the polymers include sites that will accept, bind, or otherwise combine with the antimicrobial material to more integrally incorporate the antimicrobial material into the chemical structure of the fiber.

The antimicrobial material used with the fiber can be provided in a variety of embodiments. Generally, the antimicrobial material should be provided to the fiber in a manner such that the antimicrobial material is physically positioned to exert its antimicrobial properties on the surrounding environment, such as near a wound. Accordingly, the antimicrobial material can be included in the inventive fibers in any fashion wherein an effective antimicrobial activity can be achieved. Without intending to be bound by theory, the ability of the fibers of the invention to wick fluids improves the delivery of the antimicrobial material to the fluid-generating surface, e.g., to a wound of a human or other animal.

In specific embodiments, the antimicrobial material is incorporated into the polymeric material used to form the grooved fiber. For example, the antimicrobial material could be combined with the molten polymer immediately prior to spinning the fibers. In this manner, the spun fiber includes the antimicrobial material as an integral part of the fiber body. Preferably, the antimicrobial material is substantially uniformly distributed throughout the grooved fiber. In particular, the antimicrobial material is distributed throughout the fiber such that at least a portion of the antimicrobial material is present at the outer surface of the fiber. This is particularly beneficial in that the uniformly distributed antimicrobial material is present at the outer fiber surface across the entire extent of the surface. Preferably, an antimicrobial material will be chosen such that the antimicrobial material will migrate to the polymer surface at least one of during extrusion and after extrusion. In one embodiment, the antimicrobial material is added to the polymeric material in a concentrated form, mixed with the polymeric material, and extruded.

In an embodiment of the invention, an antimicrobial material is included as part of a coating that is applied to the grooved fiber. Any process known in the art for coating compositions onto the surface of a fiber may be used. For example, thermal spray processes are characterized by the steps of first heating up the coating material and then accelerating the material towards the fiber using a heated gaseous medium.

In another example of a coating process, an apparatus is used to deposit a layer of an antimicrobial material comprised of an antimicrobial agent and a resin across the surface of a fiber. In this embodiment, the surface of the fiber preferably has an affinity for the resin. The resin layer allows the antimicrobial agent to become adhered to the coated surface. Of course, any other compound capable of allowing an antimicrobial agent to become adhered to a surface may be used.

In embodiments of the invention where the antimicrobial material is applied in such a way that it is embedded in the surface of the grooved fiber, such as through suffusion coating, a resin or other type of compound is not necessarily required to hold the antimicrobial agent in place though may be desired, in certain embodiments of the invention, to impart a greater degree of adhesion of the antimicrobial material to the grooved fiber.

In yet other embodiments of the invention, the coating may be applied at least one of substantially contemporaneously with and some time following extruding the grooved fiber.

In another embodiment of the invention, an antimicrobial material can be blended with the polymeric material used to form the grooved fiber, as described herein, and an antimicrobial material further can be included as part of a coating that is applied to the grooved fiber (e.g., after extrusion of the fiber), as also described herein. The antimicrobial material of the coating can be comprised of an antimicrobial agent that is either the same or different from an antimicrobial agent of the antimicrobial material blended with polymeric material used to form the grooved fiber.

The terms “incorporate”, “incorporates” or “incorporated,” as used herein with respect to the antimicrobial material and the grooved fiber, are intended to encompass any mode of combination or application such that a finished grooved fiber comprises the polymeric material used to form the fiber and the antimicrobial material. In other words, the terms can be construed as blending the antimicrobial material with the polymeric material used to form the grooved fiber, applying the antimicrobial material to the grooved fiber as a coating, embedding the antimicrobial material in a surface of the fiber, applying an antimicrobial material to the grooved fiber in any other way as could be contemplated by a person of ordinary skill in the art having the benefit of this disclosure, and any combination thereof.

Preferably, the grooved fibers of the invention are designed to have a requisite type and amount of an antimicrobial agent needed to impart a therapeutically effective amount of antimicrobial activity to the grooved fiber or fabrics made therefrom. As used herein, the term “therapeutically effective amount,” when referring to the antimicrobial agent of the invention, means that amount of an antimicrobial agent that elicits a desired biological or medicinal response in a tissue system of a subject, or in a subject, that is being sought by a researcher, veterinarian, medical doctor, or other clinician. The desired response includes the ability to inhibit, decrease, and/or eliminate the replication or growth of a microbial species at the site of or in the vicinity of a wound. One skilled in the art will recognize that the “therapeutically effective amount” of an antimicrobial agent to be used in the instant invention can vary with factors, such as, for example, the particular subject, e.g., age, weight, diet, health, etc.; severity of and complications from the wound being treated; the particular antimicrobial agent or antimicrobial agents used; environmental factors, such as, for example, temperature, barometric pressure, and humidity; and the amount of time that has elapsed since the wound occurred and treatment or even between successive treatments.

Any material known to be useful as an antimicrobial can be used according to certain embodiments of the invention. For example, certain metals are particularly known to exhibit useful antimicrobial activity, such as silver. Nonlimiting examples of further antimicrobial additives can be found in U.S. Pat. No. 4,525,410 to Hagiwara et al. (zeolite particles having a bactericidal activity, for example a bactericidal composition comprising a metal such as silver or copper), U.S. Pat. No. 4,906,466 to Edwards et al. (silver compounds, such as AgCl, AgBr, Ag₂CO₃, Ag₃PO₄, deposited on a physiologically inert particle selected from the oxides of Ti, Mg, Al, Si, Ce, Hf, Nb, and Ta also comprising calcium hydroxyapatite and barium sulfate with the composition optionally including a dispersion agent), and U.S. Pat. No. 6,887,270 to Miller et al. (chlorhexidien, salicylic acid, and triclosan), all of which are incorporated herein by reference.

In one embodiment of the invention, an antimicrobial material useful in the inventive fibers is a nanoparticulate material and, more preferably, the nanoparticulate material is a nanoparticle silver.

Further examples of antimicrobial materials useful in the invention include fungicides or bactericides. Nonlimiting examples include antimicrobial additives such as the silver compounds disclosed in U.S. Pat. No. 4,906,466, as well as others disclosed in U.S. Pat. No. 4,582,052 to Dunn et al. (iodine), U.S. Pat. No. 4,842,592 to Jordan et al. (cetylperidinium chloride), U.S. Pat. No. 5,620,738 (ether-based compounds), U.S. Pat. No. 6,921,546 to Albach (silver nitrate and copper nitrate), and U.S. Pat. No. 7,105,500 to Mao et al. (halogeno-o-hydroxydiphenyl compounds or non-halogenated hydroxydiphenyl ether compounds; phenol derivatives; benzyl alcohols; chlorohexidine and derivatives thereof; C₁₂₋₁₄ alkybetaines and C₈ to C₁₈ fatty acid amidoaklylbetaines; amphoteric surfactants; trihalocarbanilides; quaternary and polyquatemary compounds; and thiazole compounds), all of the foresaid patents being incorporated herein by reference.

In another embodiment of the invention, the fiber can comprise a material useful to promote the hydrophilic capability of the invention fibers. In one embodiment of the invention, the fiber comprises a surfactant to render the polymer of the fiber more wettable. In another embodiment, the surfactant can include, for example, at least one of polyoxyethylene, lauryl ether, polyoxyethylene oleyl ether, polyoxylene-polyoxypropylene-sorbitan linoleic phthalic ester, Milease T, and a potassium lauryl phosphate based lubricant.

In another embodiment of the invention, the antimicrobial material is disposed within the grooves of the fiber. Preferably, the grooves are dimensioned to be a capillary-grooved fiber. The antimicrobial material may be either partially or substantially disposed within the grooves of the capillary-grooved fiber. FIG. 6 is one example of this embodiment showing a cross-section of the capillary-grooved fiber 10 with grooves 11-18 and the antimicrobial material 20 disposed therein. In this embodiment of the invention, the antimicrobial material may be a non-leaching antimicrobial agent such as 2,4,4′-trichloro-2′-hydroxy diephenol ether or 5-chloro-2-phenol(2,4-dichlorophenoxy), the latter sold under the trademark MICROBAN® Additive B by Microban Products Company of Huntersville, N.C.

In another embodiment of the invention, other antimicrobial materials may be disposed in the fiber grooves, including antimicrobial agents in the form of a gel. Nonlimiting examples of antimicrobial gels are disclosed in U.S. Pat. No. 5,244,667 to Hagiware et al. (an antimicrobial coat of aluminosilicate on the surface of a silica gel), U.S. Pat. No. 6,800,278 to Perrault et al. (antimicrobial hydrogel formed by the polymerization of quaternary ammonium monomers in an aqueous media), and U.S. Pat. No. 6,914,051 to Allen (an antimicrobial compound such as azithromycin, erythromycin, or roxithromycin in a mobilizing agent such as an organogel compound, such as pluronic lecithin liposomal organogel), all of which are incorporated herein by reference.

In an embodiment of the present invention, the antimicrobial material disposed within the fiber grooves comprises a water soluble material and an antimicrobial agent. Without intending to be bound by theory, the water soluble material will dissolve as a hydrous fluid contacts the antimicrobial material as the hydrous fluid is wicked through a fibrous medium. The remaining antimicrobial agent can become part of the water-based fluid diffusing throughout the fluid, or, in the case of a hydrophobic antimicrobial material, be free to independently move away from the groove as the water-based fluid preferentially fills the groove through capillary action. Water soluble materials that would allow the antimicrobial agent to be disposed within the fiber grooves can comprise at least one or more of acrylate and derivatives, albumin, alginates, carbomers, carrageenan, cellulose and derivatives, dextran, dextrin, gelatin, polyvinylpyrrolidone, and starch. Examples of water soluble gels include, but are not limited to, sorbitol, glycerin, and hydroxethylcellulose. Non-limiting examples of water soluble polymers include polyvinyl alcohol, polyvinyl pyrrolidone, starch, methyl cellulose, carboxymethyl cellulose, and sodium alginate. Preferably, the water soluble materials are pharmaceutically accepted and/or biocompatible such as those compositions described U.S. Pat. No. 4,765,983 entitled “Adhesive Medical Tapes for Oral Mucosa” to Takayanagi et al., U.S. Pat. No. 5,362,424 entitled “Microencapsulation for Controlled Oral Drug Delivery System” to Lee et al., U.S. Pat. No. 4,876,125 entitled “Medical Instrument and Method for Making” to Takemura et al., and U.S. Pat. No. 6,509,038 entitled “Antifungal Compositions with Improved Bioavailability” to Baert, all incorporated herein by reference.

Examples of hydrophobic antimicrobial agents can include the antimicrobial agents that are crosslinked into a carboxymethyl cellulose as disclosed in U.S. Pat. No. 5,709,870 entitled “Antimicrobial Agent” to Yoshimura et al, macroporous crosslinked polymers having an antimicrobial agent as disclosed in U.S. Pat. No. 5,145,685 entitled “Skin Treatment Method and Composition” to Carmody, or mixtures that are not susceptible to being washed away such as those disclosed in U.S. Pat. No. 5,607,683 entitled “Antimicrobial Compositions Useful for Medical Applications” to Capelli.

The antimicrobial material may be disposed in the fiber grooves by any of a number of techniques known in the art. In a simple embodiment, particularly when the grooved fibers are capillary-grooved fibers, the grooved fiber is merely drawn through a bath of antimicrobial material where the antimicrobial material is wicked into the grooves of the grooved fiber. In another embodiment, the bath comprises water, an antimicrobial material, and a surfactant, the bath being maintained at a viscosity that allows the bath fluid composition to be wicked by the fiber. Optionally, the fiber leaving the bath may be subjected to drying by any technique known to a person having skill in the art.

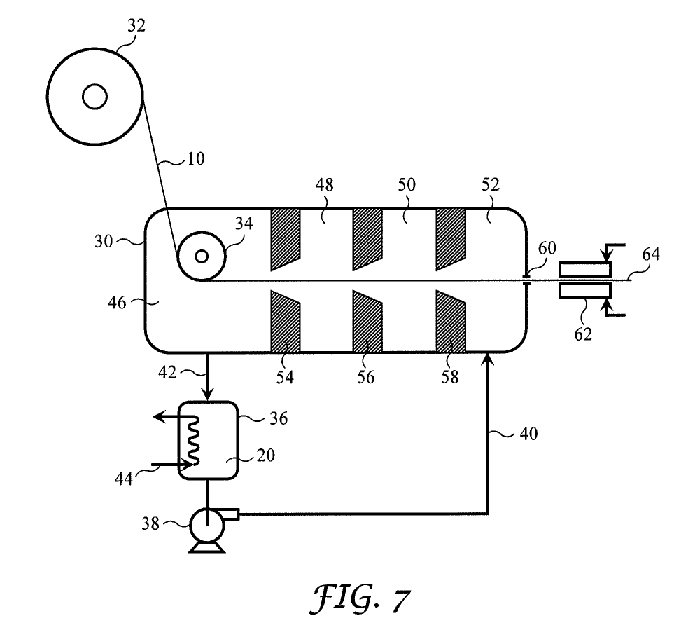

One exemplary process is illustrated in FIG. 7. The grooved fiber 10 is directed to the permeation chamber 30 by a feed roller 32, and positioned within the permeation chamber 30 by the guide roller 34. The antimicrobial material 20 is kept in the holding vessel 36 and circulated to the permeation chamber 30 by the pump 38 through the feed line 40. In this embodiment, the antimicrobial material 20 is flowing countercurrent to the direction of the grooved fiber 10. The antimicrobial material 20 that does not permeate the grooves of the grooved fiber 10 is returned to the holding vessel 36 through the recirculation line 42. The antimicrobial material may 20 optionally be heated by the heat exchange system 44. In a preferred embodiment of the invention, the antimicrobial material 20 is a viscous gel under ambient conditions allowing it to remain contained within the grooves of the grooved fiber until perhaps becoming contacted with the wicked fluid. Within this embodiment, the antimicrobial material 20 becomes less viscous at increased temperatures allowing the antimicrobial material 20 to be more easily circulated through the permeation chamber 30. The permeation chamber 30 has a forward section 46, a first and second intermediate section 48 & 50, and an aft section 52. The grooved fiber 10 is drawn through three tapering conical dies 54-58, which separate the sections 46-52, causing the antimicrobial material 20, flowing in the countercurrent direction, to be compressed against the grooved fiber 10. The grooved fiber 10 exits the permeation chamber 30 through a stripping orifice 60 causing any excess antimicrobial material 20 to be wiped away from the grooved fiber 10. As the grooved fiber 10 exits the permeation chamber, it may optionally pass through a cooling system 62. In one embodiment of the invention, the cooling system 62 is comprised of air that passes over the treated capillary grooved fiber 64. In another embodiment of the invention, the cooling system 62 is comprised of a chilled cooler. The grooved fiber 10 has been permeated with antimicrobial material 20 to produce a finished fluid wicking, antimicrobial fiber 64 according to these described embodiments of the invention.

In specific embodiments, the grooved fiber may be a multicomponent fiber. For example, the fiber could be in the form of a sheath/core fiber, wherein the core comprises a first polymer composition and the sheath comprises a second polymer composition. In preferred embodiments, the second polymer composition (forming the sheath) comprises an antimicrobial material, as described herein. Such embodiments are particularly useful in that the overall amount of antimicrobial material included in the fiber can be reduced, thus reducing the overall cost of the fiber. The first polymer composition and the second polymer composition may comprise any of the polymers described herein and may be the same or different. For example, in one embodiment, the first polymer composition and the second polymer composition could comprise the same polymer and differ only in that the second polymer composition comprises the antimicrobial material. In other embodiments, the first polymer composition could comprise a polymer that is different from a polymer of which the second polymer composition is comprised.

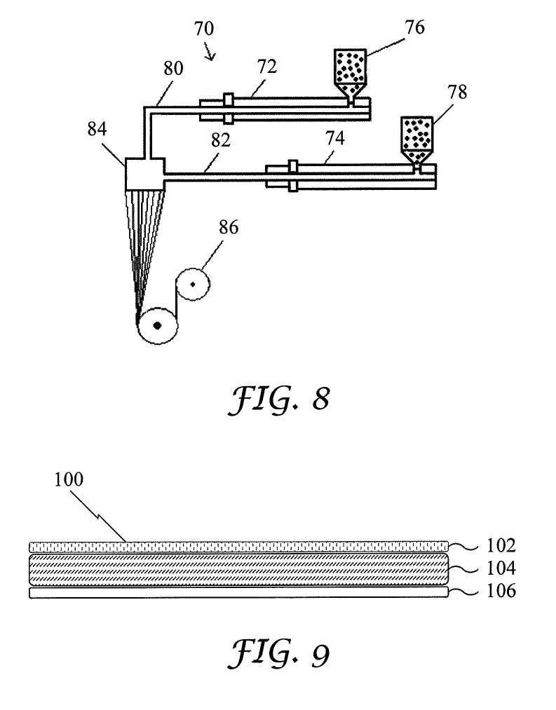

A fiber according to the invention, including a multicomponent fiber, can be prepared using any of the fiber formation techniques as known in the art. An exemplary method for producing a multicomponent fiber is illustrated in FIG. 8, which illustrates a melt spinning line 70 for producing bicomponent fibers, and which includes a pair of extruders 72 and 74. As will be appreciated by the skilled artisan, additional extruders may be added to increase the number of components (for example, wherein a plurality of temperature-regulating inner fiber components are encapsulated by an outer fiber component in a sheath/core embodiment). Moreover, a similar process could be used for extruding a single component fiber using only a single extruder.

In FIG. 8, extruders 72 and 74 separately extrude a first fiber component and a second fiber component. The first fiber component is fed into extruder 72 from a hopper 76 and the second fiber component is fed into extruder 74 from a separate hopper 78. The first fiber component and the second fiber component are fed from extruders 72 and 74 through respective conduits 80 and 82 by a melt pump (not shown) to a spinneret 84.

The separate fiber components are preferably matched to allow spinning of the components through a common capillary at substantially the same temperature without degrading one of the components. The invention, however, should not be viewed as limited to combinations of fiber components with substantially similar extrusion temperatures.

Extrusion processes and equipment, including spinnerets, for making multicomponent continuous filament fibers are well known and need not be described here in detail. Generally, a spinneret includes a housing containing a spin pack which includes a plurality of plates stacked one on top of the other with a pattern of openings arranged to create flow paths for directing fiber-forming components separately through the spinneret. The spinneret has openings or holes arranged in one or more rows. The polymers are combined in a spinneret hole. The spinneret is configured so that the extrudant has the desired overall fiber cross section (e.g., round, trilobal, etc.). The spinneret openings form a downwardly extending curtain of filaments. Such a process and apparatus is described, for example, in U.S. Pat. No. 5,162,074, to Hills, which is incorporated herein by reference.

Following extrusion through the die, the resulting thin fluid strands, or filaments, remain molten for some distance before they are solidified by cooling in a surrounding fluid medium, which may be chilled air blown through the strands (not shown). Once solidified, the filaments are taken up on a godet or other take-up surface. For example, in a continuous filament process as illustrated in FIG. 8, the strands are taken up on godet rolls 86 that draw down the thin fluid streams in proportion to the speed of the take-up godet.

The fibers of the present invention can be used in their filament form, or they could be formed into staple fibers, spunbond, or melt-blown to form fabrics, or the like. Accordingly, in another aspect, the present invention provides a fabric at least partially comprising a fiber as described herein. Fabrics encompassed by the present invention include, without limitation, nonwoven fabrics, woven fabrics, and knit fabrics. Fibers that are not cut (filament yarns) may be formed into fabrics by knitting or weaving, optionally in combination with other yarns. Staple fibers may be spun, optionally in combination with other staple fibers, into spun yarns. These yarns can be formed into fabrics by knitting or weaving. Staple fibers, optionally in combination with other staple fibers, also may be formed into nonwoven fabrics by wet-laid processes, such as paper-forming, by air-laid processes, or by carding to form a card web that can be subsequently strengthened by thermal bonding, chemical bonding, needlepunching, stitchbonding or hydroentangling.

The fiber of the invention can be incorporated into various fabrics, as described above, in varying amounts, depending upon the desired properties of the fabric. In certain embodiments, fabrics according to the invention can comprise from about 1% by weight to 100% by weight of the inventive fiber disclosed herein. In further embodiment, the inventive fabric can comprise about 5% to 100% by weight, about 10% to 100%, about 20% to 100%, about 30% to 100%, about 40% to 100%, about 50% to 100%, about 60% to 100%, about 70% to 100%, about 80% to 100%, or about 90% to 100% by weight of the inventive fiber.

Another aspect of the invention includes products made from the inventive fabrics and/or inventive fibers disclosed herein. In an embodiment of the invention, the product comprising the fabric as disclosed herein is provided. In another embodiment of the invention, as illustrated in FIG. 9, an antimicrobial absorbent structure 100 is generally comprised of three layers—a liquid pervious layer 102, a core structure 104, and a liquid impervious layer 106. The liquid pervious layer 102 allows the passage of a fluid to the core structure 104 while the liquid impervious layer 106 contains the fluid preventing it from leaving the antimicrobial absorbent structure 100. The fluid-wicking, antimicrobial fibers as disclosed herein can be found in the core structure 104. Further, there is an advantage, in some embodiments of the invention, in incorporating the fibers of the invention in at least one of the liquid pervious layer 102 and the liquid impervious layer 106. In one embodiment, the function of the liquid pervious layer 102 is to collect and distribute the fluid to the core structure 104. In this regard, the fibers of the invention may have some advantage in certain embodiments since they offer the ability to wick the fluid away from the surface and would allow the antimicrobial material to be placed closer to surface when the antimicrobial absorbent structure 100 is first applied. The general antimicrobial absorbent structure 100 of FIG. 9 can be used in, but not limited to, products including diapers, incontinence pads, sanitary napkins, certain articles of clothing, cosmetic appliances, wound dressings, and other personal hygiene products such as skin cleansing applicators, and the like.

As disclosed herein, the core structure 104 may comprise the fluid-wicking, antimicrobial fibers of the invention that are in the form of a woven, a nonwoven fabric, a knitted fabric, a filament yarn, a tow, a staple fiber, or combinations thereof. Indeed, the core structure 104 may itself comprise one or more layers.

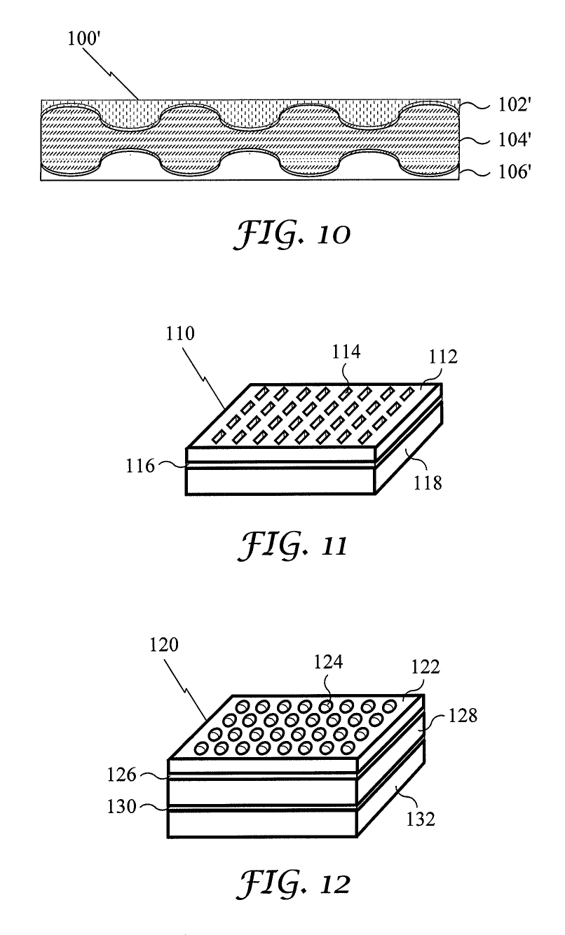

Other embodiments with different geometrical cross-sectional shapes can be contemplated by a person having skill in the art based upon this disclosure. For example, FIG. 10 shows another antimicrobial absorbent structure 100′ having a liquid pervious layer 102′, a core structure 104′, and a liquid impervious layer 106′ with the liquid pervious layer 102′ and liquid impervious layer 104′ having shapes to mate with the shape of the core structure 104′ as provided in this embodiment. Nonlimiting purposes for forming the core structure 104′ as provided in this embodiment include providing increased structural support to the antimicrobial absorbent structure 100′, increasing the volume of the core structure 104′ for the same applied unit surface area of the antimicrobial absorbent structure 100′, and enhancing the comfort of the antimicrobial absorbent structure 100′. Other conformations of the layers of the antimicrobial absorbent structure 100′ can be predicted by persons of ordinary skill in the art for meeting any number of purposes of products that comprise the fluid-wicking, antimicrobial fibers of the current invention. Such conformations are intended to be part of the disclosure herein.

The antimicrobial absorbent structure of the invention is characterized by its ability to wick fluids away from a surface, its high absorbency to store said fluids within its fibrous structure, and its microbicidal functionality-i.e., its ability to inhibit, reduce, or even eliminate microbes and to deliver an antimicrobial material to the surface of the antimicrobial absorbent structure as needed.

The products of the invention may comprise an antimicrobial absorbent structure as merely one component. As those familiar with the art understand, absorbency and the antimicrobial capability are only two features that such products are designed to deliver. Additional features may be designed into a product comprising the antimicrobial absorbent structure of the disclosure herein for the purpose of making the product beneficial for its intended purpose. Products comprising the antimicrobial absorbent structure as disclosed herein including such additional design features are intended to be part of this disclosure. Such additional design features include, but are not limited to, additional cover sheet materials such as those disclosed in U.S. Pat. No. 6,867,344 to Potts et al.; a dressing support layer for the wound dressing of U.S. Pat. No. 6,838,589 to Liedtke; a shape and size to make the product inconspicuous as disclosed in U.S. Pat. No. 6,749,594 to Hansson et al.; bandages with thermal inserts disclosed in U.S. Pat. No. 6,599,266 to Masini; an adhesive and/or attachment means such as those disclosed in U.S. Pat. No. 6,805,961 to Watanabe et al., U.S. Pat. No. 6,255,553 to Sullivan, and U.S. Pat. No. 5,100,399 to Janson et al.; and modifying the density and composition of fibers within such structures to control the rate of absorbency and the rate of fluid flux that is the subject of the disclosure in U.S. Pat. No. 4,610,678 to Weisman et al. and as further disclosed herein. In fact, the art is replete with many such features all of which are intended to be encompassed as part of the inventions that are the subject of this disclosure.

Another embodiment of the invention is a medical device comprising the fluid-wicking, antimicrobial fibers as disclosed herein. In certain embodiments, the disclosed devices can restore the transepithelial skin capability; maintain proper moisture control at the site of a wound; create a microbial barrier; inhibit, reduce, or even eliminate microbial production at the site of or in the vicinity of a wound; deliver an antimicrobial material to a wound; reduce pain; and combinations thereof. In an embodiment of the invention, the medical device is a wound dressing. The wound dressings of the invention comprise one or more layers of materials. At least one of the one or more layers is comprised of the fluid-wicking, antimicrobial fibers as further disclosed herein. Certain embodiments of the invention provide devices or dressings having the proper fiber configurations and densities such that various moisture levels and rates of fluid flux can be achieved, depending on any number of properties including, but not limited to, size of wound, type of wound, fluid-generation capability of a human or other animal, as well as any other therapeutic consideration.

FIG. 11 shows an exemplary multilayer wound dressing device. The wound dressing 110 of the illustration is comprised of a substrate 112 and substantially rectangular apertures 114. An adhesive 116 attaches the substrate 112 to an absorbent layer 118. The absorbent layer 118 comprises the fluid-wicking, antimicrobial fibers as further disclosed herein. Additionally, the substrate 112 may also comprise the fluid-wicking, antimicrobial fibers as further disclosed herein, or alternatively, and more preferably in this case, the substrate 112 will comprise an inventive fabric of the present disclosure.

At least one embodiment of the present invention provides a medical device comprising at least one layer of conformable material with such conformable material comprising the fluid-wicking, antimicrobial fibers of the present invention and/or fabrics made therefrom. The conformable material generally has the ability to wick fluids and retain said fluids within the grooves of the inventive fibers. The conformable material may have a plurality of layers including at least one layer comprising the inventive fibers as disclosed herein. The device can have at least one layer, at least two layers, at least three layers, at least four layers, at least five layers, at least six layers, at least seven layers, at least eight layers, at least nine layers, at least ten layers, and more.

Any layer of the medical device of the invention may comprise at least one fabric of the invention as further disclosed herein. The fabric can be a woven fabric, a nonwoven fabric, a knitted fabric, and combinations thereof. In other embodiments, at least one layer of the medical device of the invention comprises the fluid-wicking, antimicrobial fibers of the invention as further described herein, with the inventive fibers in the form of at least one of a filament yarn, a tow, and a staple fiber.

An embodiment of the invention provides a medical device comprising a fluid-wicking, antimicrobial fiber of the invention. The fluid wicking, antimicrobial fiber is comprised of grooved fibers substantially free of an antimicrobial material.

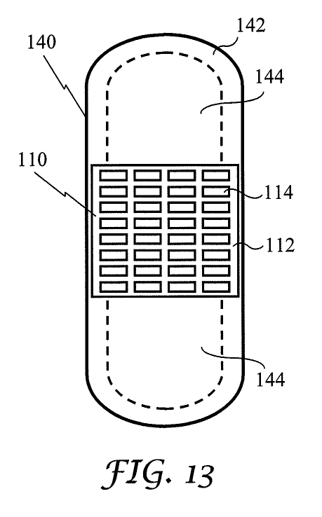

FIG. 12 shows an embodiment of a wound dressing 120 having a substrate 122 with substantially circular apertures 124. An adhesive 126 attaches the substrate 122 to a first absorbent layer 128. A second absorbent layer 132 is attached to the first absorbent layer 128 by another attachment means 130. In one example of this embodiment, the first absorbent layer 128 comprises the fluid-wicking, antimicrobial fibers as further disclosed herein and the second absorbent layer 132 comprises grooved fibers, preferably capillary-grooved fibers, substantially free of an antimicrobial material. In another example of this embodiment, the first absorbent layer 128 comprises grooved fibers, preferably capillary-grooved fibers, substantially free of an antimicrobial material and the second absorbent layer 132 comprises the fluid-wicking, antimicrobial fibers as further disclosed herein. In further examples of the embodiment as illustrated in FIG. 12, the substrate 122 may comprise at least one of the fluid-wicking, antimicrobial fibers as further disclosed herein and capillary-grooved fibers substantially free of an antimicrobial material. In the embodiment where the substrate 122 is comprised of an antimicrobial material, the substrate more preferably comprises an inventive fabric as further disclosed herein.

In embodiments where there are multiple layers, one skilled in the art with the benefit of this disclosure can comprehend any number of configurations where certain layers contain at least one of the fluid-wicking, antimicrobial fibers as further described herein.

As appreciated by a person skilled in the art with the insight provided by this disclosure, the inventive wound dressings may have a range of mechanical properties depending on, among other things, the type of polymer selected, the size and distribution of fibers within any one of the layers of the medical device, and the types of adhesive that may optionally be used in the construction of the device.

As appreciated by a person skilled in the art with the insight provided by this disclosure, the inventive wound dressings may comprise materials that allow the medical device to provide comfort when applied to the wound of a human or other animal. Other embodiments of the invention provide a wound dressing that further comprises additives to reduce pain and/or provide comfort to the wound bearing human and/or animal. Yet other embodiments of the invention provide additional compositions to further assist the wound healing process.

Another aspect of the invention is a bandage that is comprised of at least one of the inventive wound dressings as further disclosed herein. FIG. 13 shows an embodiment of a bandage 140 that is comprised of the wound dressing 110 shown in FIG. 11. The bandage of this embodiment further comprising a contact surface 142 and an adhesive 144 that, in this embodiment, is positioned in two diametrically opposite sections relative to the wound dressing 110. Optionally, the substrate 112 and/or the contact surface 142 may include a hypoallergenic layer (not shown) in the event the human and/or animal is sensitive to the material of which the substrate 112 and/or contact surface 142 is constructed. With the benefit of this disclosure, one skilled in the art can comprehend many other uses of the inventive wound dressings that are disclosed herein. Such embodiments are intended to be a part of the disclosure herein.

Another aspect of the invention is a method of increasing the healing rate of a wound comprising the steps of having an exposed wound surface, covering the exposed wound surface with a fabric or device comprising a grooved fiber according to the invention. In another embodiment, the method for increasing the healing rate of a wound further comprises the step of maintaining the original covering, wound dressing, and the like on the exposed wound surface until the wound has healed.

All publications mentioned herein, including patents, patent applications, and journal articles are incorporated herein by reference in their entireties including the references cited therein, which are also incorporated herein by reference. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It will be appreciated by those skilled in the art that changes could be made to the embodiments described herein without departing from the broad inventive concept thereof. Therefore, it is understood that this invention is not limited to the particular embodiments disclosed, but it is intended to cover modifications within the spirit and scope of the present invention as defined by the appended claims.

That which is claimed:

1. A fluid-wicking, antimicrobial fiber comprising a grooved fiber having an outer fiber surface and having a cross-section that forms continuous longitudinal grooves along the fiber, wherein the fiber is formed of a polymeric material and incorporates an antimicrobial material such that at least a portion of the antimicrobial material is in contact with the outer fiber surface.

2. The fiber of claim 1, wherein the antimicrobial material comprises a metal.

3. The fiber of claim 2, wherein the metal comprises silver.

4. The fiber of claim 1, wherein the antimicrobial material comprises a nanoparticulate material.

5. The fiber of claim 1, wherein the polymeric material comprises a melt-spinnable thermoplastic.

6. The fiber of claim 1, wherein the polymeric material comprises at least one material selected from the group consisting of nylon 6, nylon 6,6, polyethylene terephthalate, polybutylene terephthalate, polypropylene terephthalate, polylactic acid, polypropylene, polyethylene, and combinations thereof.

7. The fiber of claim 1, wherein the antimicrobial material is blended with the polymeric material.

8. The fiber of claim 1, wherein grooved fiber is a multicomponent fiber.

9. The fiber of claim 8, wherein the multicomponent fiber is in the form of a sheath/core fiber formed of a sheath component and a core component.

10. The fiber of claim 9, wherein the sheath component comprises the antimicrobial material.

11. The fiber of claim 10, wherein the antimicrobial material is blended with a polymeric material used to form the sheath component.

12. A fabric comprising a fiber according to claim 1.

13. The fabric of claim 12, wherein the fabric is selected from the group consisting of a woven fabric, a nonwoven fabric, a knitted fabric, and combinations thereof.

14 The fabric of claim 12, wherein the fiber is in the form of a filament yarn, tow, or staple fibers.

15. A wound dressing comprising a fabric according to claim 12.

16. A wound dressing comprising:
an adhesive portion for adhering to an area of skin adjacent a wound; and
a fabric for covering an exposed wound surface, the fabric comprising a grooved fiber having an outer fiber surface and having a cross-section that forms continuous longitudinal grooves along the fiber, wherein the fiber is formed of a polymeric material and incorporates an antimicrobial material such that at least a portion of the antimicrobial material is in contact with the outer fiber surface;
wherein the grooved fiber wicks fluid away from the wound surface while simultaneously reducing or eliminating microbe viability at the wound surface.
